(19) United States
(12) Patent Application Publication
(10) Pub. No.: US 2019/0038192 A1
(43) Pub. Date: Feb. 7, 2019

(54) TREATMENT PROTOCOL SYSTEM WITH ORAL HYGIENE DEVICE AND RELATED METHODS

(71) Applicant: Winter Lion Healthcare Instruments, Inc., Winter Park, FL (US)

(72) Inventors: Eric Randolph Miller, Winter Park, FL (US); Manon Chin, Orlando, FL (US); Chris Donald Mason, Saint Cloud, FL (US)

(21) Appl. No.: 16/156,222

(22) Filed: Oct. 10, 2018

Related U.S. Application Data
(63) Continuation-in-part of application No. 15/813,940, filed on Nov. 15, 2017.
(60) Provisional application No. 62/422,749, filed on Nov. 16, 2016.

(51) Int. Cl. A61B 5/145; A61C 19/04; A61B 17/24; A61B 5/00

(52) U.S. Cl. CPC A61B 5/14539; A61B 5/14507; A61C 19/04; A61N 5/0624; A61B 5/682; A61B 5/4836; A61B 17/24

(57) ABSTRACT

A treatment protocol system may include oral hygiene devices. Each oral hygiene device may detect a pH level within an opening of the user/animal, perform a comparison of the pH level within the opening of the user/animal with a threshold pH level, and selectively control a pH neutralization device based upon the comparison. The treatment protocol system also may have a server to receive a plurality of treatment parameters from each oral hygiene device. The include the pH level from each oral hygiene device, a neutralization agent use level from each oral hygiene device, and a health characteristic from an associated patient for each oral hygiene device. The server may be configured to generate a pH treatment protocol for the associated patient for each oral hygiene device based upon the treatment parameters.

### RELATED APPLICATION

This application is a continuation in part of U.S. patent application Ser. No. 15/813,940 filed Nov. 15, 2017 which claims benefit of U.S. Application No. 62/422,749 filed Nov. 16, 2016, the entire subject matters of which are incorporated herein by reference in their entireties.

### TECHNICAL FIELD

The present disclosure relates to the field of electronic devices, and, more particularly, to an oral hygiene device and related methods.

### SUMMARY

Generally, a treatment protocol system may include a plurality of oral hygiene devices. Each oral hygiene device may comprise a housing configured to be inserted into an opening of a user/animal, a processor carried by the housing, a pH sensor carried by the housing, and a pH neutralization device carried by the housing and configured to emit neutralization agent into the opening of the user/animal, the processor coupled to the pH sensor and the pH neutralization device. The processor may be configured to detect a pH level within the opening of the user/animal, perform a comparison of the pH level within the opening of the user/animal with a threshold pH level, and selectively control the pH neutralization device based upon the comparison. The treatment protocol system also may comprise a server comprising a processor, and a memory cooperating therewith and configured to receive a plurality of treatment parameters from each oral hygiene device. The plurality of treatment parameters may include the pH level from each oral hygiene device, a neutralization agent use level from each oral hygiene device, and at least one health characteristic from an associated patient for each oral hygiene device. The server may be configured to generate a pH treatment protocol for the associated patient for each oral hygiene device based upon the plurality of treatment parameters.

In some embodiments, each oral hygiene device may comprise an image sensor device coupled to the processor and configured to generate imaging data from the opening of the user/animal. The plurality of treatment parameters may include the imaging data. Also, the server may be configured to execute a supervised machine learning process on the plurality of treatment parameters to generate the pH treatment protocol.

Moreover, the processor may be configured to generate opening position data associated with the pH level. Each oral hygiene device may include at least one additional biosensor carried by the housing. Each oral hygiene device may include a power supply coupled to the processor, and an activation switch coupled to the processor. Each oral hygiene device may include an output nozzle configured to emit the neutralization agent into the opening of the user/animal. Each oral hygiene device may comprise a wireless transceiver coupled to the processor and configured to connect to an external network for data transmission to the server. Each oral hygiene device may include a neutralization process indicator configured to generate an indication of progress in a neutralization process.

Another aspect is directed to a method for operating treatment protocol system comprising a server, and a plurality of oral hygiene devices. Each oral hygiene device may include a housing configured to be inserted into an opening of a user/animal, a processor carried by the housing, a pH sensor carried by the housing, and a pH neutralization device carried by the housing and configured to emit neutralization agent into the opening of the user/animal. The method may comprise operating the plurality of oral hygiene devices to detect a pH level within the opening of the user/animal, perform a comparison of the pH level within the opening of the user/animal with a threshold pH level, and selectively control the pH neutralization device based upon the comparison. The method may also include operating the server in communication with the plurality of oral hygiene devices. The server is to receive a plurality of treatment parameters from each oral hygiene device. The plurality of treatment parameters may include the pH level from each oral hygiene device, a neutralization agent use level from each oral hygiene device, and at least one health characteristic from an associated patient for each oral hygiene device. The server may also generate a pH treatment protocol for the associated patient for each oral hygiene device based upon the plurality of treatment parameters.

### BRIEF DESCRIPTION OF THE DRAWINGS

FIG. 1 is a schematic diagram of an oral hygiene device, according to the present disclosure.

FIG. 2 is a schematic top plan view of a first embodiment of the oral hygiene device, according to the present disclosure.

FIG. 3 is a schematic side elevational view of the oral hygiene device of FIG. 2.

FIG. 4 is a flowchart of a method for operating the oral hygiene device of FIGS. 2-3.

FIG. 5 is a schematic top plan view of a second embodiment of the oral hygiene device.

FIG. 6 is a schematic diagram of a second embodiment of the oral hygiene device.

### DETAILED DESCRIPTION

The present disclosure will now be described more fully hereinafter with reference to the accompanying drawings, in which several embodiments of the invention are shown. This present disclosure may, however, be embodied in many different forms and should not be construed as limited to the embodiments set forth herein. Rather, these embodiments are provided so that this disclosure will be thorough and complete, and will fully convey the scope of the present disclosure to those skilled in the art. Like numbers refer to like elements throughout, and base 100 reference numerals are used to indicate similar elements in alternative embodiments.

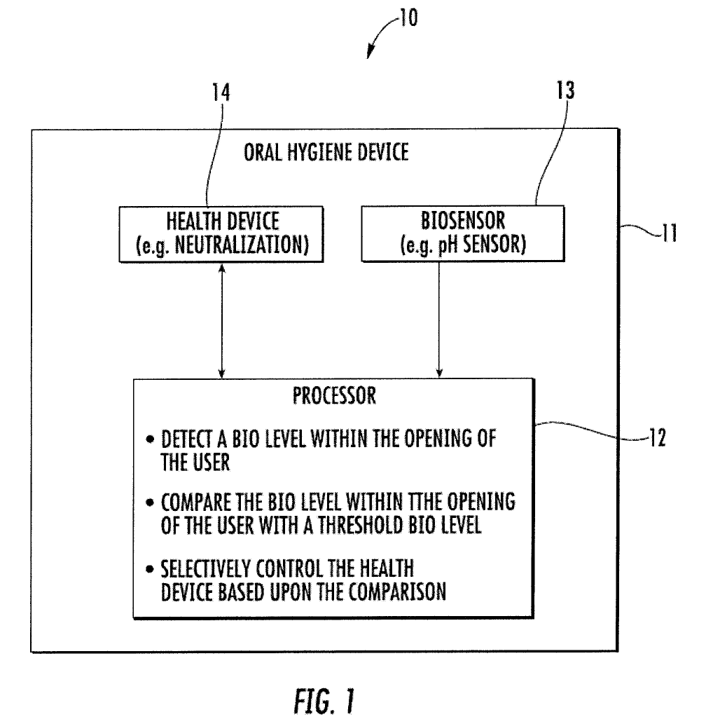

Referring initially to FIG. 1, an oral hygiene device 10 according to the present disclosure is now described. The oral hygiene device 10 illustratively includes a housing (e.g. an elongate housing) 11 having first and second ends. The second end is configured to be inserted into an opening of a user/animal, and the oral hygiene device 10 provides for maintenance of the pH levels (among other biological values) within the opening of the user/animal (i.e. controlling the acidity/basicity within the opening of the user/animal).

The oral hygiene device 10 illustratively includes a processor (e.g. system-on-chip processing integrated circuit) 12 carried by the housing 11. The oral hygiene device 10 illustratively includes a biosensor (e.g. a pH sensor) 13 carried by the housing 11 at the second end.

In some embodiments, the oral hygiene device 10 illustratively includes one or more additional biosensors. The additional biosensors may provide biological data related to pH levels, but may provide a very different data value, such as, for example, temperature, bacteria presence, deficiencies in normal values, inflammation, decay, infections, allergies and other health conditions. Also, the biosensors could detect analytes in saliva, breath, tissue, teeth, gums, micron and nano sized particles, which is not an exhaustive list and is provided only for exemplary purposes. In some embodiments, the biosensor 13 may comprise a multi-reading biosensor.

The oral hygiene device 10 illustratively includes a health device 14 carried by the housing 11. In some embodiments, the health device 14 may comprise a neutralization device and configured to emit a pH level neutralization agent into the opening of the user/animal. In certain embodiments, the health device 14 may comprise a health cartridge configured to emit agents, such as neutralization agents, good bacteria (probiotics) agents, inflammation reduction agents, hormone agents, vitamin agents, and other agents for health markers/needs or concerns that are detected by a sensor collected in the targeted orifice or dermatitis (skin). The agents will have varying flavor profiles. The agents may include pharmaceutical grade products, prescription or over the counter drugs, natural blends, vitamins, minerals, botanicals, liposomes, nano and micron formulations, energetics, veterinary formulas, hormones and other health targeted solutions. Of course, this list of agents is not exhaustive, and other agents could be used. Also, the health device 14 uses a variable or controlled release agent to optimize the timing of remediation process to most efficiently rectify the health condition.

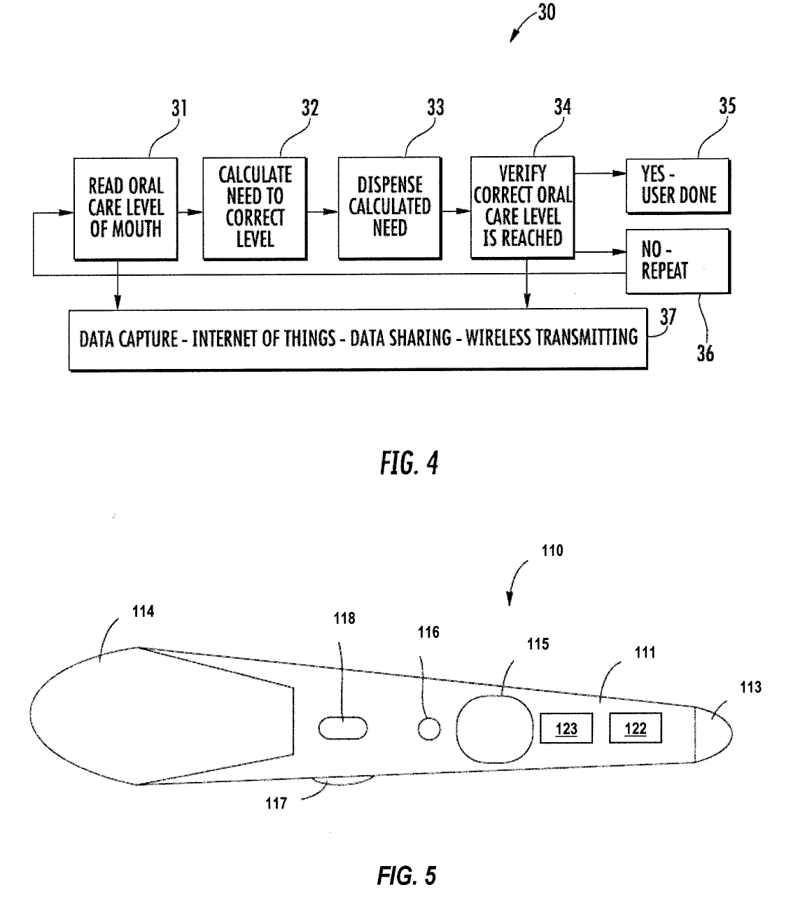

In some embodiments (e.g. FIGS. 3-4), the health device 14 is replaceable/interchangeable. For example, the health device 14 could be swapped out to provide varying sensing capabilities. In yet other embodiments, the health device 14 is static or fixed.

The processor 12 is coupled to the pH sensor 13 and the health device 14. The processor 12 is configured to detect a biological value (e.g. pH level) within the opening of the user/animal, compare the biological value within the opening of the user/animal with a threshold biological value, and selectively control the health device 14 based upon the comparison. In some embodiments, the biological value within the opening of the user/animal is compared to a desired biological value range.

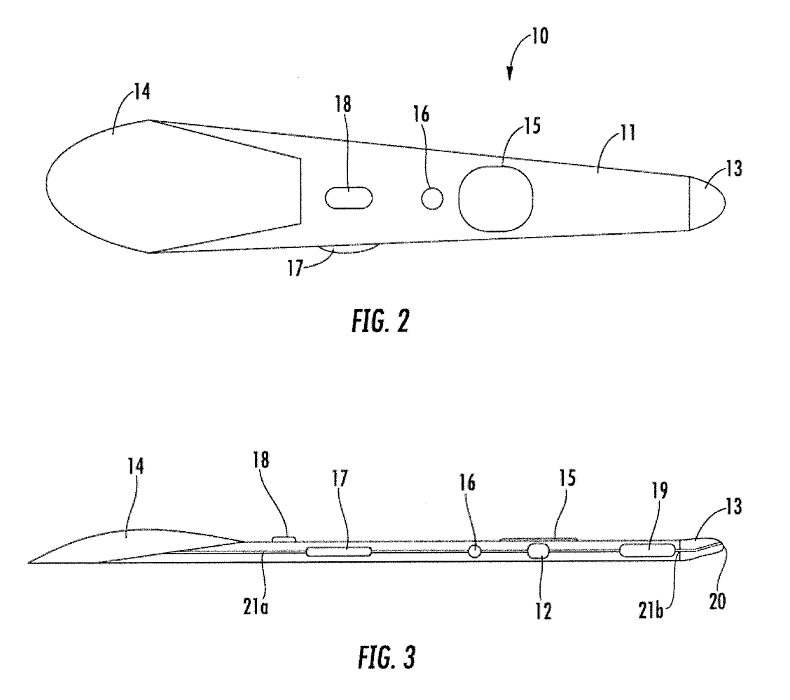

Referring now additionally to FIGS. 2-3, in the illustrated oral hygiene device 10, the health device 14 illustratively includes a pH neutralization device. The oral hygiene device 10 illustratively includes a pH range indicator 15 configured to generate an indication when the pH level within the opening of the user/animal is within a pH level range. In this embodiment, the pH range indicator 15 comprises a multi-color indicator (e.g. a multi-color light emitting diode (LED) module) to indicate whether the pH level within the opening of the user/animal is above, below, or within a desired pH level range. In this embodiment, the pH sensor 13 comprises a calibration free pH sensor. Also, this embodiment is intended for use in the oral opening of the user/animal, i.e. the mouth, but in other embodiments, the oral hygiene device 10 can be used in other bodily openings, such as the nasal opening, the vaginal opening, the ear opening, and the rectal opening, thereby providing a generic hygiene device. Also, it should be appreciated that the oral hygiene device 10 could be used on animals for similar functionality.

The oral hygiene device 10 illustratively includes a power supply 16 coupled to the processor 12, and an activation switch 17 coupled to the processor. The power supply 16 may comprise a replaceable battery power source, for example, and should provide several months or more of operation. Other embodiments may include an internal rechargeable battery source. The activation switch 17 is configured to receive input from the user, which causes the processor 12 to start the neutralization process. In this embodiment, the activation switch 17 comprises a soft-tip switch, for example, a rubberized toggle switch. The oral hygiene device 10 illustratively includes a neutralization process indicator (e.g. multi-color LED module) 18 configured to generate an indication when the neutralization process is in process and/or complete.

The oral hygiene device 10 illustratively includes an output nozzle (e.g. atomizer spray nozzle) 20 configured to emit the neutralization agent into the opening of the user/animal, and a pump device 19 coupled to the power supply 16 and configured to pressurize/direct the neutralization agent to the output nozzle. The oral hygiene device 10 illustratively includes first and second conduits 21a-21b coupled between the pH neutralization device 14 and the output nozzle 20 and providing fluid communication therebetween.

In certain embodiments, the pH neutralization device 14 comprises a replaceable pH neutralization cartridge, which is attached via a mechanical interlocking feature. In other embodiments, the pH neutralization device 14 may comprise a neutralization agent reservoir/tank/module, which may be refillable.

In some embodiments, the second end of the elongate housing 11 is replaceable for commercial application, i.e. high traffic use. The replaceable portion of the second end of the elongate housing 11 includes only the output nozzle 20 and a portion of the second conduit 21b, keeping per use costs down. The replaceable portion of the second end of the elongate housing 11 illustratively includes an interlocking feature, permitting a tight mechanical attachment to the first end of the elongate housing.

In some embodiments, the oral hygiene device 10 may comprise a wireless transceiver (e.g. IEEE 802.11 transceiver, a Bluetooth transceiver, or a near field communications (NFC) transceiver) (not shown) configured to connect to an external network (e.g. the Internet) for data transmission to web services. In some applications, the pH level data can be stored in the web services.

In other embodiments, the oral hygiene device 10 may comprise a wired data interface (not shown) coupled to the processor 12, and in rechargeable battery embodiments, the power supply 16. The wired data interface may comprise a universal serial bus (USB) interface, such as micro USB, and the wired data interface is configured to permit a wired connection to the processor 12. Via either the wired data interface or the wireless transceiver, the processor 12 may receive software updates to the neutralization algorithm.

The pH range indicator 15 and the neutralization process indicator 18 may also generate audio indications. In wireless embodiments, the indicators may send wireless alerts to external devices, such as generating a notification on an associate cellular device.

Referring now additionally to FIG. 4, a flowchart 30 illustrates a method for operating the oral hygiene device 10. The method includes inserting the second end of the elongate housing 11 into the opening of the user/animal (Blocks 31, 37), and operating the pH sensor 13 and the processor 12 for detecting a pH level within the opening of the user/animal. The method includes operating the processor 12 to process the pH level within the opening of the user/animal with a neutralization algorithm to determine if a neutralization process is needed, and if so, determining an amount of neutralization agent to be dispensed (Block 32).

The method includes operating the processor 12 and the pump device 19 to emit the amount of the neutralization agent into the opening of the user/animal (Block 33). The method also includes operating the pH sensor 13 and the processor 12 for detecting a new pH level within the opening of the user/animal (Block 34), and operating the processor to process the new pH level within the opening of the user/animal with the neutralization algorithm to determine if an additional neutralization process is needed (Block 35). The processor 12 is configured to restart the neutralization process if the additional neutralization process is needed, and to otherwise, end the neutralization process (Block 36).

Referring now additionally to FIG. 5, another embodiment of the oral hygiene device 110 is now described. In this embodiment of the oral hygiene device 110, those elements already discussed above with respect to FIGS. 1-4 are incremented by 100 and most require no further discussion herein. This embodiment differs from the previous embodiment in that this oral hygiene device 110 illustratively includes an image sensor device 122 (e.g. complementary metal-oxide-semiconductor (CMOS) integrated circuit device) coupled to the processor (not shown) and configured to generate imaging data from the opening of the user/animal. The image sensor device 122 may comprise a thermal (i.e. infrared) image sensor, a visible light image sensor, a digital image sensor, or a scanning image sensor, for example. Also, this oral hygiene device 110 illustratively includes a gyroscope 123 coupled to the processor. The processor may cooperate with the gyroscope 123 to generate opening position data associated with the pH level. In particular, the position data may be used to approximate a location of the biosensor 113 in the opening of the user/animal. Of course, in other embodiments, the gyroscope 123 may be omitted, and the position data can be provided via alternative arrangements.

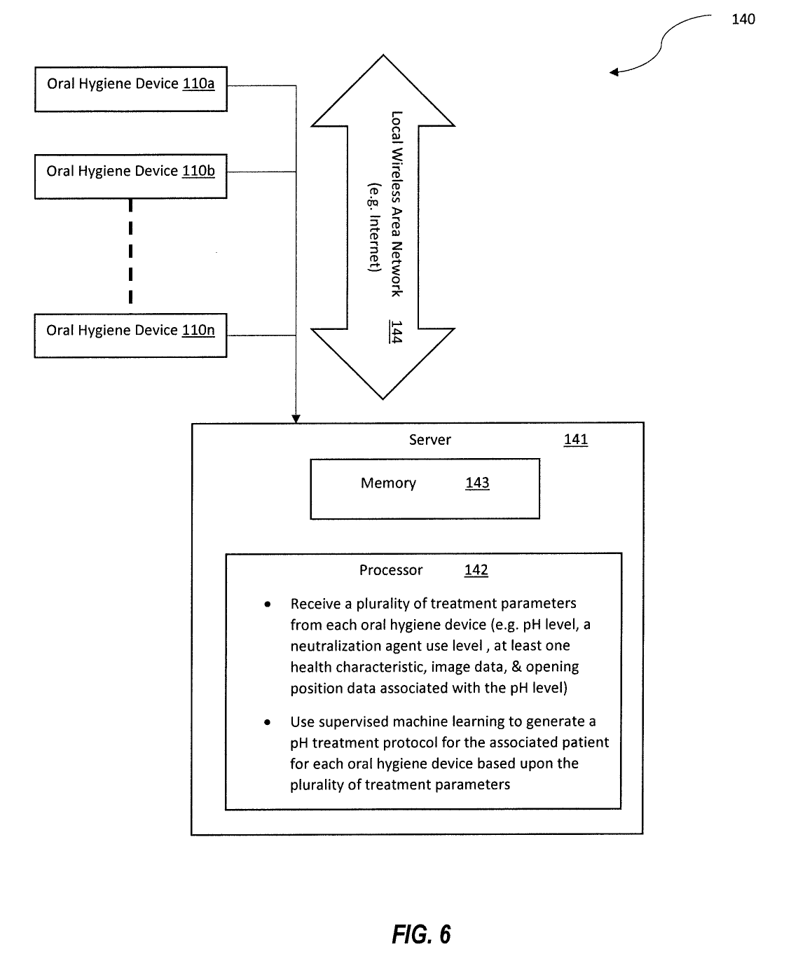

Referring additionally to FIG. 6, a treatment protocol system 140 according to the present disclosure is now described. The treatment protocol system 140 illustratively includes a plurality of oral hygiene devices 110a-110n. For example, in this illustrated embodiment, the plurality of oral hygiene devices 110a-110n each comprises the embodiment of FIG. 5, but of course, other embodiments disclosed herein could be used in the treatment protocol system 140. The treatment protocol system 140 illustratively includes a server 141 comprising a processor 142, and a memory 143 cooperating therewith and configured to receive a plurality of treatment parameters from each oral hygiene device 110a-110n. The plurality of oral hygiene devices 110a-110n are in communication with the server 141 over the local area network (LAN) 144, for example, a closed internal network or the Internet.

The plurality of treatment parameters may include the pH level from each oral hygiene device 110a-110n (including raw pH level readings, daily pH level mean, weekly pH level mean), a neutralization agent use level from each oral hygiene device, at least one health characteristic from an associated patient for each oral hygiene device, imaging data from the opening of the user/animal, and opening position data associated with the pH level. In short, the oral hygiene device 110a-110n maps the deployment of neutralization agent in the opening, and the reading of all data. Moreover, when data is generated by the oral hygiene device 110a-110n, the server 141 is configured to generate time and date values associated with the readings.

In some embodiments, the plurality of treatment parameters may also include age and gender of the associated patient for each oral hygiene device 110a-110n, and one or more lifestyle characteristics of the of the associated patient for each oral hygiene device 110a-110n (e.g. smoker, drinker, sleep patterns, education, single/married, and residential zip code).

The server 141 is configured to generate a pH treatment protocol for the associated patient for each oral hygiene device 110a-110n based upon the plurality of treatment parameters. In some embodiments, the server 141 is configured to execute a supervised machine learning process on the plurality of treatment parameters to generate the pH treatment protocol.

In some embodiments, the treatment protocol system 140 includes a plurality of wireless communications devices (not shown, e.g. cellular devices, tablet computing devices) associated with the patient. In this embodiment, each mobile wireless communications device is configured to execute a software application communication with the server 141. The server 141 is configured to communicate the treatment protocol for the patient to the associated mobile wireless communications device.

In some embodiments, the machine learning is configured to generate association rules between the plurality of treatment parameters, and overall health outcomes. In particular, some research shows a correlation between the pH level of the oral cavity and incidence of hospital-acquired infections (HAI).

Advantageously, the pH treatment protocol is proactive relative to current data points from the plurality of oral hygiene devices 110a-110n; the pH treatment protocol is focused on creating a benchmark defined in daily pH means; the pH treatment protocol establishes probability standards via historical science and evidence weighted against daily pH means; the pH treatment protocol establishes benchmarks weighted against individual patient, short, mid-term and long term goals; the pH treatment protocol weighs pH averages to future health conditions via probability and consequence (e.g. HAI, pneumonia); the pH treatment protocol weighs and compares means pH to same and different demographics, locales, use and consistency of use, time of use, and lifestyle; and the pH treatment protocol is conveyed in real time to the individual patient and or professional practitioner.

Another aspect is directed to a method for operating treatment protocol system 140 comprising a server 141, and a plurality of oral hygiene devices 110a-110n. Each oral hygiene device 110a-110n includes a housing 111 configured to be inserted into an opening of a user/animal, a processor carried by the housing, a pH sensor 113 carried by the housing, and a pH neutralization device 114 carried by the housing and configured to emit neutralization agent into the opening of the user/animal. The method comprises operating the plurality of oral hygiene devices 110a-110n to detect a pH level within the opening of the user/animal, perform a comparison of the pH level within the opening of the user/animal with a threshold pH level, and selectively control the pH neutralization device 114 based upon the comparison. The method also includes operating the server 141 in communication with the plurality of oral hygiene devices 110a-110n. The server 141 is to receive a plurality of treatment parameters from each oral hygiene device 110a-110n. The plurality of treatment parameters includes the pH level from each oral hygiene device 110a-110n, a neutralization agent use level from each oral hygiene device, and at least one health characteristic from an associated patient for each oral hygiene device. The server 141 also generates a pH treatment protocol for the associated patient for each oral hygiene device 110a-110n based upon the plurality of treatment parameters.

Many modifications and other embodiments of the present disclosure will come to the mind of one skilled in the art having the benefit of the teachings presented in the foregoing descriptions and the associated drawings. Therefore, it is understood that the present disclosure is not to be limited to the specific embodiments disclosed, and that modifications and embodiments are intended to be included within the scope of the appended claims.

That which is claimed is:

1. A treatment protocol system comprising:
a plurality of oral hygiene devices, each oral hygiene device comprising
a housing configured to be inserted into an opening of a user/animal,
a processor carried by said housing,
a pH sensor carried by said housing, and
a pH neutralization device carried by said housing and configured to emit neutralization agent into the opening of the user/animal,
said processor coupled to said pH sensor and said pH neutralization device, said processor configured to
detect a pH level within the opening of the user/animal,
perform a comparison of the pH level within the opening of the user/animal with a threshold pH level, and
selectively control the pH neutralization device based upon the comparison; and
a server comprising a processor, and a memory cooperating therewith and configured to
receive a plurality of treatment parameters from each oral hygiene device, the plurality of treatment parameters comprising the pH level from each oral hygiene device, a neutralization agent use level from each oral hygiene device, and at least one health characteristic from an associated patient for each oral hygiene device, and
generate a pH treatment protocol for the associated patient for each oral hygiene device based upon the plurality of treatment parameters.

2. The treatment protocol system of claim 1 wherein each oral hygiene device comprises an image sensor device coupled to said processor and configured to generate imaging data from the opening of the user/animal; and wherein the plurality of treatment parameters comprises the imaging data.

3. The treatment protocol system of claim 1 wherein said server is configured to execute a supervised machine learning process on the plurality of treatment parameters to generate the pH treatment protocol.

4. The treatment protocol system of claim 1 wherein said processor is configured to generate opening position data associated with the pH level.

5. The treatment protocol system of claim 1 wherein each oral hygiene device comprises at least one additional biosensor carried by said housing.

6. The treatment protocol system of claim 1 wherein each oral hygiene device comprises a power supply coupled to said processor, and an activation switch coupled to said processor.

7. The treatment protocol system of claim 1 wherein each oral hygiene device comprises an output nozzle configured to emit the neutralization agent into the opening of the user/animal.

8. The treatment protocol system of claim 1 wherein each oral hygiene device comprises a wireless transceiver coupled to said processor and configured to connect to an external network for data transmission to said server.

9. The treatment protocol system of claim 1 wherein each oral hygiene device comprises a neutralization process indicator configured to generate an indication of progress in a neutralization process.

10. A treatment protocol system comprising:
a plurality of oral hygiene devices, each oral hygiene device comprising
a housing configured to be inserted into an opening of a user/animal,
a processor carried by said housing,
a pH sensor carried by said housing,
a pH neutralization device carried by said housing and configured to emit neutralization agent into the opening of the user/animal, and
an image sensor device configured to generate imaging data from the opening of the user/animal
said processor coupled to said pH sensor, said pH neutralization device, and said image sensor device, said processor configured to
detect a pH level within the opening of the user/animal,
perform a comparison of the pH level within the opening of the user/animal with a threshold pH level, and
selectively control the pH neutralization device based upon the comparison; and
a server comprising a processor, and a memory cooperating therewith and configured to
receive a plurality of treatment parameters from each oral hygiene device, the plurality of treatment parameters comprising the pH level from each oral hygiene device, a neutralization agent use level from each oral hygiene device, at least one health characteristic from an associated patient for each oral hygiene device, and the imaging data, and
execute a supervised machine learning process on the plurality of treatment parameters to generate a pH treatment protocol for the associated patient for each oral hygiene device based upon the plurality of treatment parameters.

11. The treatment protocol system of claim 10 wherein said processor is configured to generate opening position data associated with the pH level.

12. The treatment protocol system of claim 10 wherein each oral hygiene device comprises at least one additional biosensor carried by said housing.

13. The treatment protocol system of claim 10 wherein each oral hygiene device comprises a power supply coupled to said processor, and an activation switch coupled to said processor.

14. The treatment protocol system of claim 10 wherein each oral hygiene device comprises an output nozzle configured to emit the neutralization agent into the opening of the user/animal.

15. The treatment protocol system of claim 10 wherein each oral hygiene device comprises a wireless transceiver coupled to said processor and configured to connect to an external network for data transmission to said server.

16. The treatment protocol system of claim 10 wherein each oral hygiene device comprises a neutralization process indicator configured to generate an indication of progress in a neutralization process.

17. A method for operating treatment protocol system comprising a server, and a plurality of oral hygiene devices, each oral hygiene device comprising a housing configured to be inserted into an opening of a user/animal, a processor carried by the housing, a pH sensor carried by the housing, and a pH neutralization device carried by the housing and configured to emit neutralization agent into the opening of the user/animal, the method comprising:
operating the plurality of oral hygiene devices to
detect a pH level within the opening of the user/animal,
perform a comparison of the pH level within the opening of the user/animal with a threshold pH level, and
selectively control the pH neutralization device based upon the comparison; and
operating the server in communication with the plurality of oral hygiene devices to
receive a plurality of treatment parameters from each oral hygiene device, the plurality of treatment parameters comprising the pH level from each oral hygiene device, a neutralization agent use level from each oral hygiene device, and at least one health characteristic from an associated patient for each oral hygiene device, and
generate a pH treatment protocol for the associated patient for each oral hygiene device based upon the plurality of treatment parameters.

18. The method of claim 17 wherein each oral hygiene device comprises an image sensor device coupled to the processor and configured to generate imaging data from the opening of the user/animal; and wherein the plurality of treatment parameters comprises the imaging data.

19. The method of claim 17 further comprising operating the server to execute a supervised machine learning process on the plurality of treatment parameters to generate the pH treatment protocol.

20. The method of claim 17 further comprising operating the plurality of oral hygiene devices to generate opening position data associated with the pH level.
